(19) United States
(12) Patent Application Publication
(10) Pub. No.: US 2017/0020438 A1
(43) Pub. Date: Jan. 26, 2017

(54) SYSTEM AND METHOD FOR MONITORING USER POSTURE

(71) Applicant: Monash University, Clayton, Victoria (AU)

(72) Inventor: Stephen Jia Wang, Clayton (AU)

(21) Appl. No.: 15/302,364

(22) PCT Filed: May 2, 2014
(86) PCT No.: PCT/AU2014/000488

§ 371 Date: Oct. 6, 2016

(30) Foreign Application Priority Data
Apr. 15, 2014 (AU) 2014901381

(51) Int. Cl. A61B 5/00; G01L 1/00; G06F 3/01; A61B 5/103; A61B 5/117

(52) U.S. Cl. CPC A61B 5/4561; A61B 5/1036; A61B 5/117; A61B 5/6891; A61B 5/0022; A61B 5/743; G06F 3/011; G01L 1/00; A61B 2562/0247; A61B 2562/046

(57) ABSTRACT

A posture of a user is monitored by obtaining first pressure sensor information from a plurality of sensors upon a first item of furniture occupied by the user at a first time. First sensor position information defining the relative positions of the sensors upon the first item of furniture is also obtained. The first pressure sensor information and the first sensor position information are processed to determine a first user posture upon the first item of furniture. User posture is monitored over time and/or upon different items of furniture, and a cumulative load upon the user's spine over time is determined.

### CROSS-REFERENCE TO RELATED APPLICATIONS

This application is a National Phase patent application and claims the priority of International Application Number PCT/AU2014/000488, filed May 2, 2014, which claims the benefit of Australian Provisional Patent Application No. 2014901381 filed Apr. 15, 2014, the entire contents of which are incorporated herein by reference.

### TECHNICAL FIELD

The present invention relates to a system and method for monitoring an individual's posture, body position, frequency of movement and pressure distribution, by use of pressure sensors mounted as a pressure sensing matrix upon furniture occupied by the individual.

### BACKGROUND OF THE INVENTION

Sitting is one of the most common behaviours in daily life. A recent epidemiological study on about 50,000 adults from 20 countries reported sitting time was 300 minutes/day on average.

Incorrect sedentary positions and prolonged sitting has become a serious health threat to people in modern societies, and carries the risk of various spinal problems. In particular, time spent sitting is associated with premature mortality, diabetes, and risks of cardiovascular disease, and this is so irrespective of other time the person spends exercising. A large number of studies have convincingly reported the association between different levels of exposure to occupational sitting and the presence or severity of low back pain (LBP). There is also unequivocal evidence that sitting and upper quadrant musculoskeletal pain (UQMP) are related. Moreover, a clear link has been demonstrated between thoraco-lumbar postures while sitting, and head/neck posture and motor activity. The third European Working Conditions Survey identified the most common work-related health problem as backache, reported by 33% of respondents. These problems often result in absence from work or even permanent disability, and translate to high economic costs.

It is a challenge for a very large number of people to maintain appropriate sitting positions in daily life, to avoid such seating related health issues. Discomfort and pressure sores have received attention in military, workplace, assisted living and mobility contexts. However it remains a challenge to maintain appropriate sedentary behaviour to reduce extra burden on the spine and trunk muscles.

While there remains uncertainty as to what constitutes an ideal sitting posture, it is clear that fixed postures, particularly in prolonged sitting, constitute a high risk factor for developing LBP due to static loading of soft tissues and discomfort. As a preventive strategy, fixed postures in prolonged sitting should be avoided. Accordingly it is desirable to monitor a person's spinal position and spinal movements while sitting, and to prompt the person to take appropriate proactive measures when needed.

To this end, detectors can be attached to the person, however this can be time consuming, does not allow assessment of the subject in their normal environment, and is impractical as a solution for any great length of time such as the course of a day.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides a method of monitoring posture of a user, the method comprising:

obtaining first pressure sensor information from a plurality of sensors upon a first item of furniture occupied by the user at a first time;

obtaining first sensor position information defining the relative positions of the sensors upon the first item of furniture;

processing the first pressure sensor information and the first sensor position information to determine a first user posture upon the first item of furniture;

obtaining second pressure sensor information from a plurality of sensors upon a second item of furniture occupied by the user at a second time;

obtaining second sensor position information defining the relative positions of the sensors upon the second item of furniture;

processing the second pressure sensor information and the second sensor position information to determine a second user posture upon the second item of furniture; and

determining a cumulative load upon the user's spine from the first user posture and the second user posture.

According to a second aspect the present invention provides a system for monitoring posture of a user, the system comprising:

a first item of furniture bearing a first plurality of sensors configured to output first pressure sensor information;

a second item of furniture bearing a second plurality of sensors configured to output second pressure sensor information; and

at least one server computer having a microprocessor and an associated data store which contains instructions executable by the microprocessor such that the server computer is operable to:

process the first pressure sensor information and the first sensor position information to determine a first user posture upon the first item of furniture;

process the second pressure sensor information and the second sensor position information to determine a second user posture upon the second item of furniture; and

determine a cumulative load upon the user's spine from the first user posture and the second user posture.

According to a third aspect the present invention provides a furniture component for monitoring posture of a user, the furniture component comprising:

a plurality of pressure sensors to detect a pressure exerted by a user occupying the item of furniture,

communications means for communicating the sensor information, and information reflecting the relative positions of the sensors, to at least one server computer configured to determine a user posture upon the item of furniture.

Embodiments of the invention preferably further generate a graphical display indicative of the user's current posture. The graphical display may comprise an image of a chair of the type currently occupied by the user, and the graphical display may present an indication of where on the chair the user is placing pressure in their current posture. For example a colour scale may be used to indicate variations in pressure applied by the user to the chair. In some embodiments, pressure values at 0, 50 and 100 kilopascal could be presented as colour variations (i.e. yellow, blue and red colours), respectively, with intermediate values represented by intermediate colours. The colour scale of such embodiments thus may convert real-time pressure-distributions to a colour mapping. In some embodiments the hotspots on the mapping rendering may comprise each pressure sensor location, and each such hotspot may have a surrounding fade-off area defined by the amount of received pressure on the sensor.

Additionally or alternatively, in some embodiments the graphical display may comprise an image representing a human spine or vertebral column, viewed at an appropriate scale, the image generated in a manner to indicate a current posture of the user's spine as determined from the sensor data. A shading colour scale may be used to indicate in the image those portions or sections of the spine which are suffering elevated load as a result of the determined posture of the user. Such embodiments of the present invention thus provide a method and system that converts seated posture data into a 3D representation of spine position. Where the user posture is repeatedly determined over time, and the image of the spine is in turn updated repeatedly over time, such embodiments may thus provide an interactive system which displays spinal movement between different postures.

In some embodiments, the sensor position information defining the relative positions of the sensors upon the first item of furniture may be stored in a suitable data storage attached to or borne by the item of furniture, with such information being appended to the sensor information. Alternatively, the sensor position information defining the relative positions of the sensors upon the first item of furniture may comprise a furniture item identifier, which may be used to look up the physical arrangement of sensors on that item of furniture by reference to a furniture ID database of such data, the furniture ID database being separate to the item of furniture. The furniture ID database may be an online resource accessible via the Internet, for example. Such embodiments thus provide for each item of furniture involved in the system to be configured with an identifier, whereby data produced from each item of furniture can be communicated to a central processor and the central processor can determine which item of furniture produced the data, to determine the cumulative load on the user's spine.

Each determination of user posture preferably includes an accommodation of individual characteristics of the individual user. The individual characteristics might for example include user height, medication history, injuries, age, weight, and/or other information relevant to posture. The accommodation of such characteristics preferably results in the determined posture more accurately reflecting the user's actual posture. The user's individual characteristics are preferably held as user profile data in a user profile component of the system. The user profile component may be stored in the data store of the server computer, or may be stored in a separate data storage device accessible for example via the Internet. The user profile component may be password protected or otherwise secured by or on behalf of the user, to retain data privacy of the user.

In some embodiments of the invention output data including or derived from the determined posture and/or cumulative spinal load is delivered via a messaging system for review by the user or others. In such embodiments, the furniture ID database may further contain data identifying a display associated with the item of furniture occupied by the user, such that the messaging system may direct information to the associated display. For example, if the item of furniture is an office chair the furniture ID database may cause messaging to be directed to a desktop computer associated with the office chair, and if the item of furniture is an armchair the furniture ID database may cause messaging to be directed to a television associated with the armchair. Additionally or alternatively, the furniture ID database may contain data identifying a personal device associated with the user, such as a smartphone or tablet device, so as to cause messaging to be directed to the personal device of the user. Such embodiments may thus prompt the user to help prevent harmful and fixed postures in prolonged sitting. Additionally or alternatively the output data may be delivered to a physician to assist diagnosis and/or treatment of the user.

The item of furniture may be an office chair, a lounge chair, a chair of a vehicle such as a car or aeroplane, a bed, or other item of furniture. Embodiments monitoring user posture in relation to a vehicle may be particularly advantageous in monitoring instantaneous pressure, which can vary considerably when a car passes over uneven road or when an aeroplane experiences turbulence or touches down, for example. Such instantaneous loads can thus be included in the determination of the cumulative load on the user's spine.

The monitoring period may comprise a workday of the user, for example from 9 A.M. to 5 P.M. However some embodiments may alternatively provide for a monitoring period which encompasses both work times, travel times, and rest times, by providing a user's office chair, car seat and armchair with suitable sensors.

The item of furniture may further be adjustable, and equipped with sensors which indicate a current position of each adjustable element of the item of furniture. For example, a sensor may detect a current height of an adjustable-height chair, and the posture determination may thus take into account that for example when the chair is in a low position the user may exert more pressure on the chair with their ischial tuberosities at the rear of the seat than with their hamstrings at the front of the seat, as compared to when the chair is in a high position. Similarly, sensors may be provided to detect a current backrest position, seat angle, armrest height, and the like.

The furniture component of the third aspect may be the whole item of furniture or may be a sub portion of furniture such as a slip configured to be fitted upon the furniture.

According to another aspect the present invention provides a method of monitoring posture of a user, the method comprising:

obtaining pressure sensor information from a plurality of sensors upon an item of furniture occupied by the user;

obtaining sensor position information defining the relative positions of the sensors upon the item of furniture;

processing the pressure sensor information and the sensor position information to determine a user posture upon the item of furniture; and

generating a graphical representation of the user's current posture and displaying the graphical representation to the user on a display associated with the user.

According to yet another aspect the present invention provides a method of identifying a user of an item of furniture, the method comprising:

obtaining pressure sensor information from a plurality of sensors upon the item of furniture occupied by the user;

obtaining sensor position information defining the relative positions of the sensors upon the item of furniture;

processing the pressure sensor information and the sensor position information to determine a morphology of a user currently occupying the item of furniture; and

comparing the determined morphology to records of the morphology of a plurality of individuals in order to determine which one of the plurality of individuals is occupying the item of furniture.

### BRIEF DESCRIPTION OF THE DRAWINGS

An example of the invention will now be described with reference to the accompanying drawings, in which:

FIG. 1 illustrates a system of one embodiment of the present invention;

FIG. 2 illustrates the system architecture of the embodiment of FIG. 1;

FIGS. 3a and 3b illustrate a graphical display produced for a user by the embodiment of FIGS. 1 and 2;

FIG. 4 illustrates the system architecture of another embodiment;

FIG. 5 illustrates the design and minimum components of a standard sensing unit (i.e. a mat style design) in accordance with an embodiment for monitoring ‘sitting’ positions of the invention which could be used as an attachment to chairs;

FIG. 6 illustrates the design and minimum components of a standard sensing system (i.e. a mat style design) in accordance with an embodiment for monitoring ‘sleeping’ positions of the invention which could be used as an attachment to beds;

FIG. 7 illustrates the detailed layout structure of the sensing unit;

FIG. 8 illustrates the details of a power supply layer of the sensing unit;

FIG. 9 illustrates the details of a signal layer of the sensing unit;

FIG. 10 illustrates the pressure sensing unit installation details; and

FIGS. 11a-11c illustrate the details of interface design for a direct-body-position-monitoring function of the pressure-sensing embodiment of FIG. 7.

### DESCRIPTION OF THE PREFERRED EMBODIMENTS

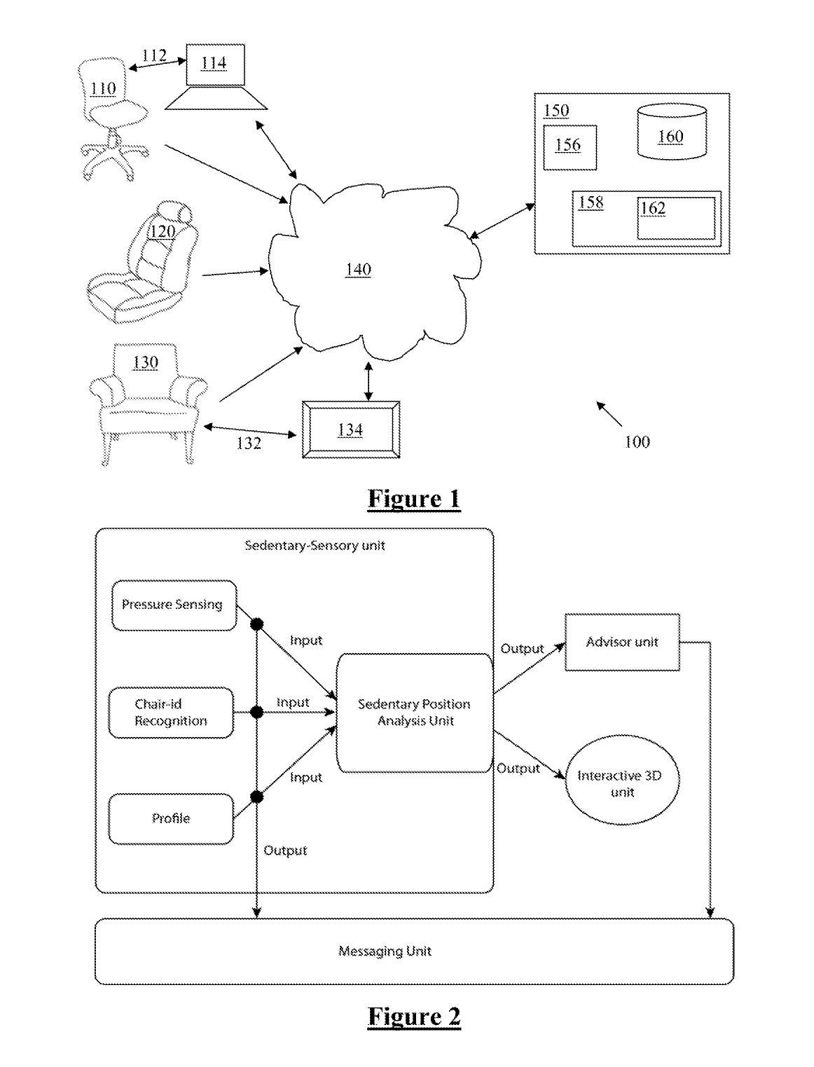

FIG. 1 illustrates a system 100 of one embodiment of the present invention. An office chair 110, car seat 120 and armchair 130 are all associated with a single user, in that on a typical day the user might drive in car seat 120 to work, spend much of the day seated on office chair 110, drive in car seat 120 to their home, and then spend some portion of the evening in armchair 130.

Server 150 is connected to the Internet 140, in a conventional manner, in order to enable remote access by users, as well as to provide the server 150 with access to related online resources. The server 150 includes at least one processor 156, which is associated with random access memory 158, used for containing program instructions and transient data related to the operation of the tasks performed by the server 150. In particular, the memory 158 contains a body of program instructions 162 implementing a method of monitoring user posture. Additionally, the body of program instructions 162 includes instructions for delivering graphical indications of posture to the user. The instructions 162 also permit the user to access such information from any device executing conventional web browser software. The processor 156 is also operatively associated with a further storage device 160 such as a hard disk drive, which is used for long-term storage of program components, as well as for storage of data relating to user posture, as well as characterising information gathered from users in the form of a user profile, as described in greater detail below. Storage device 160 also records the association between armchair 130 and TV 134, and the association between office chair 110 and desktop computer 114.

Spine movement information is collected from pressure sensors installed at various locations on the chairs 110, 120, 130. The sensor readings are combined with a respective chair ID, and a user profile to obtain contextual hypotheses. The output is delivered via a messaging system developed to help prevent harmful and fixed postures in prolonged sitting.

As shown in FIG. 2, this embodiment comprises four integrated modules a) multiple sensors, b) comprehensive data analysis (agents), c) interactive 3D (Mixed Reality), and d) messaging.

Such a system enables multiple persons to simultaneously view, discuss, and interact with the virtual 3D models, and enhance practice by supporting remote and co-located activities. The multiple sensors module monitors various data about the users. A comprehensive agent model is designed to constantly monitor users' everyday activities. Interactive 3D (Augmented Reality or MR) displays provides great flexibility of viewpoint and intuitive interfaces to present information and support users to change their behaviour. Such a Messaging system may act as a powerful persuader because it can intervene in the right context as a convenient way to prompt users to change their behaviour.

Ubiquitous computing and context-aware persuasive technologies offer an opportunity to promote healthy behaviour by presenting “just-in-time”, “appropriate time” and “appropriate place” information. The present embodiment recognises that the appropriate sedentary position varies depending on the purpose for which people sit, e.g. sitting in the office, car, dining etc. The main focus of the system design of the present embodiment is to monitor people's sedentary behaviour across various circumstances and encourage people to maintain appropriate sedentary positions under various contexts. To give location-aware advice the system relies on the “chair ID” which reflects the surrounding environment. To give advice at the right moment the system requires knowledge of the users' activities.

Advice on correct sedentary posture must fit easily into users' daily routine since messages suggesting simple activities are preferred over ones requiring significant effort. Besides, lifestyle interventions can yield positive and long-term effects, in terms of increasing levels of moderately intense physical activity. The suggested locations of lifestyle activities we include in our system are: everyday activity (shops, homes, schools, workplaces, etc.) and recreation destinations (playgrounds, parks and gardens, etc.)

The system of the present embodiment thus provides users with personalized and contextualized advice on appropriate sitting positions. The present embodiment is implemented using the Unity3D and Arduino platforms. The Unity3D engine supports exporting application to mobile platforms, which will render the 3D images in real-time based on the data from the Sedentary Position Analysis Unit.

The system architecture of this embodiment is shown in FIG. 2 and consists of a Sedentary-Sensory Unit, an advisory Unit, an Interactive 3D Unit and a messaging unit. The “Sedentary-Sensory Unit” is designed to monitor users' spine movements while sitting by detecting centre of gravity and back curvature. This unit can be designed in suitable forms, such as a pad-like device, and set up in various places to gather sedentary position information in different contexts like at the office, at home or driving. Each unit has a unique id used to recognize which chair the user is sitting on. This information is sent to the “Advisory Unit” which processes it to compute the cumulative spinal burden. Comprehensive sedentary information is presented to the user on mobile devices, computer screens or smart TVs as real-time rendered interactive 3D images. To reduce the burden on spine and motivate users to maintain healthy sedentary habits, messages are presented pervasively using the most appropriate media. For instance, advice will be displayed on TV 134 when the user is sitting on the armchair 130 or on desktop computer 114 if the user is sitting on office chair 110, or a mobile device at other times. Presentation settings could be tailored to users' privacy and other needs.

The “Sedentary-Sensory Unit” provides high-sensitivity “Pressure Sensing”, “Chair-id Recognition” and “Profile” functions. When users sit or semi-sit on the sensing pad, their body affects the pressure sensors on it. Each sensor sends the detected values as analog input, which affects the 3D visualization through the “Interactive 3D unit” to simulate the user's spine situation. Multiple physical pads should be placed on the seats habitually used for the different purposes. The pads' IDs determine the use context of each seat to provide correct posture parameters to the Sedentary Position Analysis unit. If the incorrect sedentary posture lasts over a set time threshold, the Advisor Service sends a message to the mobile UI.

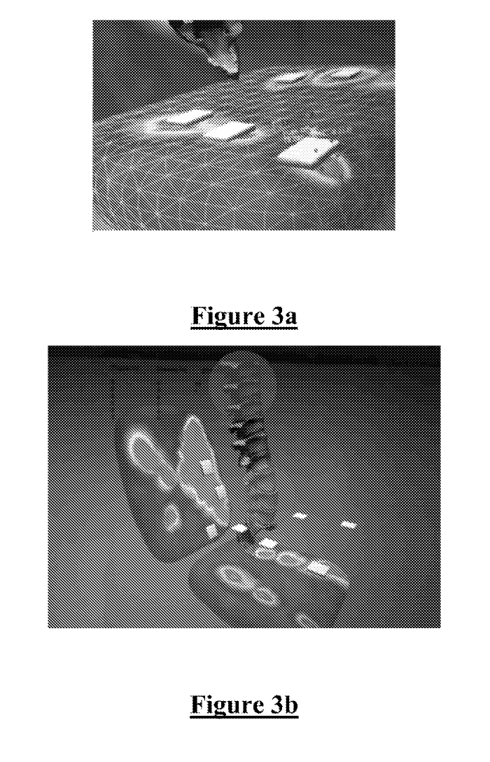

For Pressure Sensing this embodiment uses Force-Sensitive Resistor (FSR) sensors and an Arduino platform to detect postural changes. Sensors are laid out using a near-optimal sensor placement strategy. Any curvature variation of the body trunk is converted to analog input that affects the virtual 3D spine in real-time. The connection between Arduino and Unity3D is provided by a “unity-arduino serial connection”. FIG. 3 shows the “Sedentary-Sensory Unit” (left), sensors affecting virtual mat (middle) and interactive 3D spine (right).

FIG. 5 shows an example of the FSR embodiment for sitting monitoring. Where the mat 510 is designed to fit the shapes of an office chair's seat and back; the attach belt 512 is for wrapping around the back and the bottom of the chair to ensure the mat is secured to the chair surface; curved concave area 514 is designed for going around the chair back support pillar; and Velcro tapes for mat 516 and belt joint area 518 are designed for simple use, fixing and carrying.

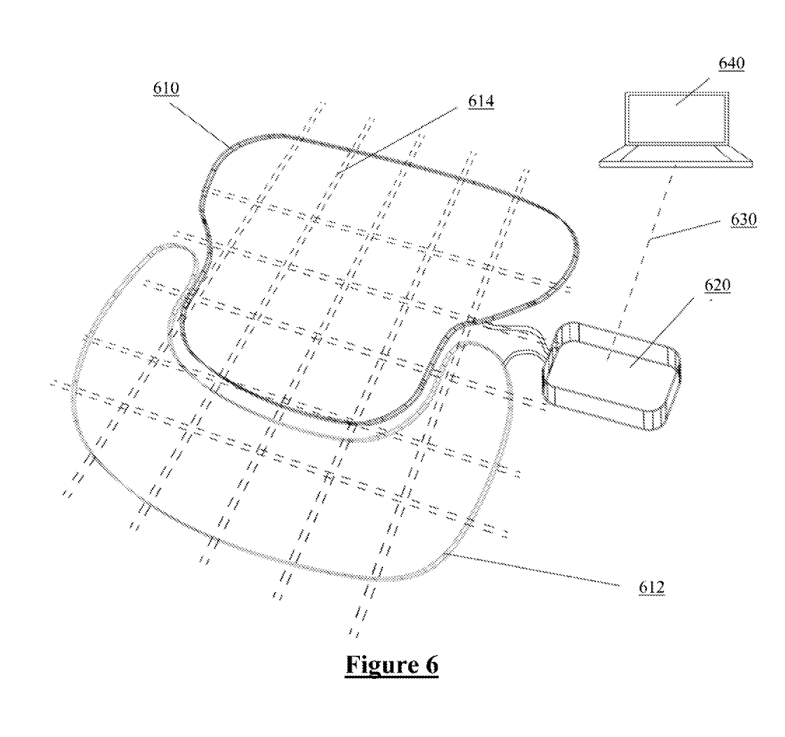

FIG. 6 shows an example of an FSR embodiment for sleep monitoring. Where the mat-upper-body 610 is designed for gathering data from back and shoulder area; mat-lower-body 612 is designed for gathering data from the sciatic area; the gap between each FSR sensing area 614 should be minimized; this unit could be connected directly to the monitoring devices 640 (i.e. computer or mobile devices) through USB cables and/or wireless connections 630 through cloud services.

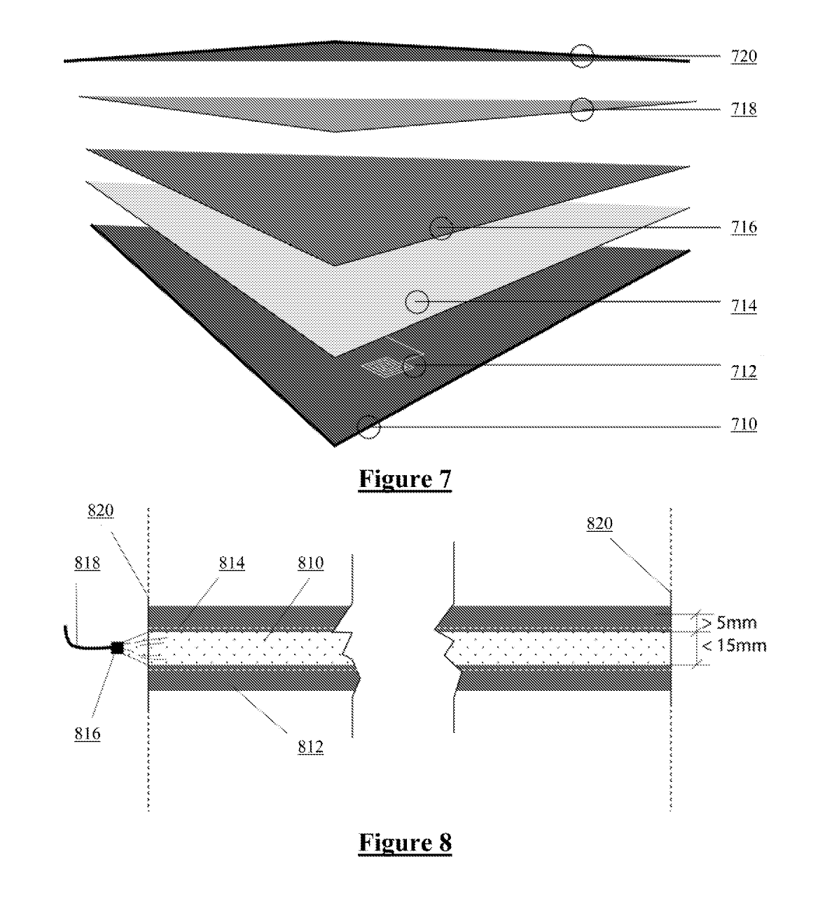

FIG. 7 illustrates the necessary structure to achieve the analogue input function and durability by using soft cloth-like materials. Where Neoprene fabric is used for the top 710 and bottom 720 outer layers of such structure; Conductive thread 712 is used to convey signals; Knitted fabric 714 (i.e. TPOLAC11IN100526 from plugandwear.com) is used to enlarge the pressure impacted area; to achieve analogue data output, Velostat conductive material (from 3M.com) 716 is used to convert the amount of pressure into a changing resistance; Knitted conductive tin copper fabric 718 is used to provide stabilized electricity power connection.

FIG. 8 shows the detailed structure of building the power-supply layer of the pressure-sensing embodiment (FIG. 7). The Knitted conductive tin copper fabric 810 is sewn on the Neoprene fabric with a certain width. In addition to this conductive material, both sides need non-conductive material 812 to achieve a strictly managed area with low-voltage (3 v-5 v) power supply; 814 are the sewing lines; this conductive fabric is connected with electrical wire 818 by soldering them together 816 outside of the edge of the Neoprene material 820.

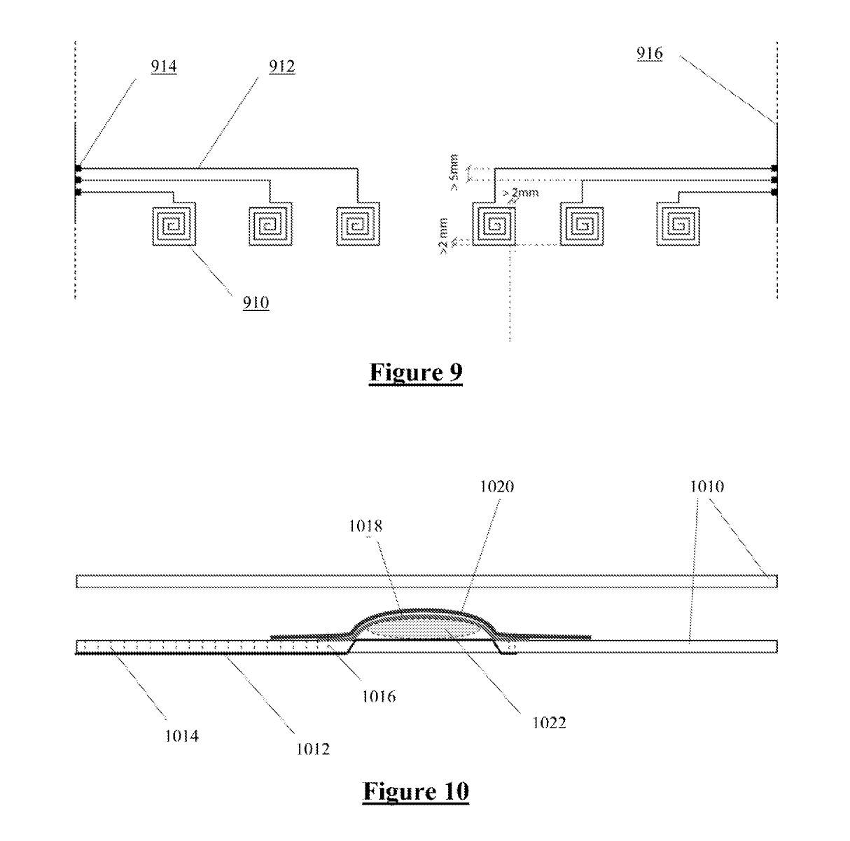

FIG. 9 shows the detailed structure of building the pressure signal-converting layer of the pressure-sensing embodiment (FIG. 7). Where the pressure-sensing is achieved by conductive thread going in helix-like shape 910 in the pressure-sensing area; except these areas, the conductive thread should go under the Neoprene material 912 to avoid miss-contacting; the end of this conductive thread needs to be sewn with little piece of conductive fabric 914 to ensure stabilized connections with electrical wires at the edge of the Neoprene fabric 916.

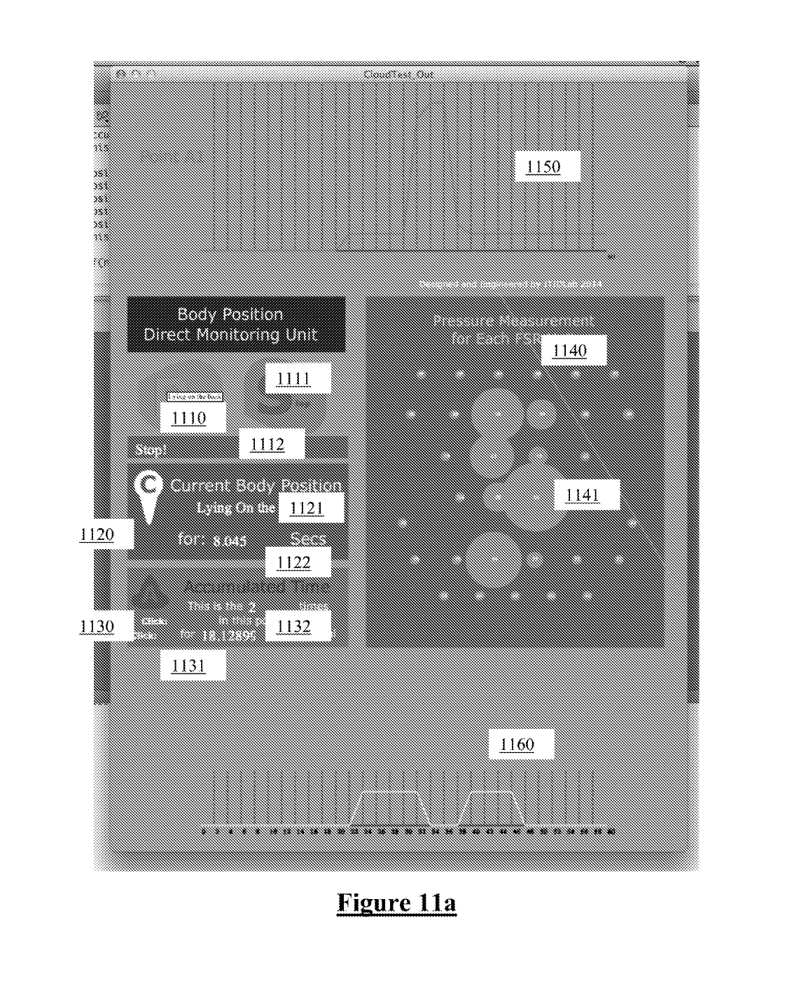

FIG. 10 shows the basic method of installing one of the pressure-sensing unit with all soft cloth-like materials to build the pressure signal-converting layer of the pressure-sensing embodiment (FIG. 7). Between the 2 Neoprene fabric 1010 the conductive threads 1012 are sewn on the Neoprene material with Non-conductive threads 1014; the data is generated by the contacts between conductive threads with the power supply layer (810 in FIG. 8a), when the conductive threads directly contact to the power supply layer (810 in FIG. 8a), the data could be received from Analog-in pins on Arduino board is 1024, without contact is 0; on top of the conductive threads, the knitted conductive fabric 1018 is used to cover a bigger area where requires pressure-sensing, the knits provide soft cushion-like press feeling; the Velostat conductive material 1020 is placed on top of the conductive fabric to facilitate analog data output through analog pins on Arduino board, the range is 0-1024; 1020 needs to fully cover (better to be bigger) than 1018; when sew these 2 conductive material onto the Neoprene material, the cavity/space needs to be constructed by the closer sewing positions 1022, which helps to avoid miss-sensing (when no-body touch the sensor, the data shows 0 or very low number output).

Functionality and ergonomic characteristics such as seat height, depth, back support angle, surface material, flexibility (adjustment), etc. for each chair are stored in a database with a unique id. The chair-ID is recognized when users sit and combined with users' profiles to calculate the most appropriate postures and sitting periods.

The Chair-ID recognition function is based on the Internet of Things technology. Chair specification could either be directly input, or acquired from the manufactures' database. In some embodiments, the latter option enables the design of smart chairs whereby ergonomic adjustments by users are monitored, and sent back to the manufacturer to inform future chair design.

Users fill in their own user profile through an online interface, thereby providing information such as body size, gender, age, common sitting time, type of transportation, etc.

The Sedentary Position Analysis Unit is to recognise unhealthy postures based on duration and detected deviations from the ideal spinal position. In the present embodiment, the ‘ideal’ sitting position is defined as being dependent on the angles of three divided spine sections: thoracic, thoraco-lumbar and lumbar. The posture detection system uses a comparison algorithm to analyze the spinal positions of each section. The chair-seat sensors are divided into four sections: a (left-front), b (left-back), c (right-front) and d (right-back) to work out the position of the lumber area. The chair-back is divided into two sections: e (left) and f (right) to calculate the position of the thoracic area. The Analysis Unit compares the pressure input from these sections to calculate the thoraco-lumbar spine section position and movement. For instance, if the value of ‘a’ is much greater than ‘c’ and ‘d’ and both e′ and ‘f’ are 0, that means the user is heavily leaning to the left-front direction. However, if the value of e′ and ‘f’ are also great at same time, that means the user is in a left leaning ‘sloppy’ position, as the thoracic area is positioned backwards.

Based on the posture recognition, chair-id and profile, the advisor service calculates a suitability score for each advice in the activity database. Advice is generated through an expert system that considers the following factors:
Spine angle: sharp spine angles cause extremely heavy burden on the spine and should not be maintained for a prolonged time
Prolonged time: this parameter measures the duration of a position Accumulated sitting time: cumulative sitting time on different chairs to calculate total sitting time
Frequency: how often the user takes this same position

Instead of monitoring how the user follows the suggested activities, this system follows a decide-choose-do format to accumulate the chosen activities into the database. The “Advisor Unit” sends a query to one or more of the services and acquires their analysis results. If all the factors are met, the advice becomes a candidate. Each item of advice mainly contains several types of message:
Warning: to alert the user it's time to change posture and stop sitting
Activity: to suggest users take a proactive relaxation approach and what kind of activity is appropriate for their context. This type of message normally gives the user several options to choose from depending on the contextual restrictions and time limitations
Relaxation: to give the option to rest rather than doing exercises; this type of message suggests a minimum time-span during which any sitting should be avoided

For example, if a user has been continuously working in his office chair for more than 3 hours, and has maintained a healthy posture for less than 20% of this period, the system of this embodiment will produce the following item of advice “Please stand up straight with your arms at your sides, bend sideways to the left, slide your left hand down your thigh and reach with your right arm over your head. Hold this position for 10 seconds, then return to the starting position and repeat for the opposite side. Alternate sides for 9 more times”. Thus, in general, embodiments of the invention may use the obtained posture data to generate suggestions of appropriate exercises which might be performed by the user.

The 3D display unit presents spine information in two modes: “real-time mode” and “accumulation mode”. The real-time mode presents the user's current spinal position and corresponding burden. The accumulation mode presents the cumulative spinal burden information gathered during a certain period. Advice is mainly generated based on the accumulation mode data. It is less often that a warning advice is directly generated from the real-time mode as this only happens when a user's movement results in an instantaneous extreme burden to the spine.

Unlike conventional location-based pervasive functions, the Messaging Unit application uses the “chair-id” to recognize users' locations. This unit interacts with users by sending messages generated by the Advisory unit through the most appropriate medium.

Thus, in this embodiment the combination of direct-sensory input, interactive 3D representation and contextual advice provides a simple but efficient method to achieve intuitive sitting support. The advice unit employs an algorithm that takes into account users' profile as well as multiple sedentary information inputs to generate contextual advice. The application can be beneficial for people who want to maintain spinal health by adopting healthy sitting habits, especially for those who seldom exercise and spend most of their time in prolonged sedentary positions.

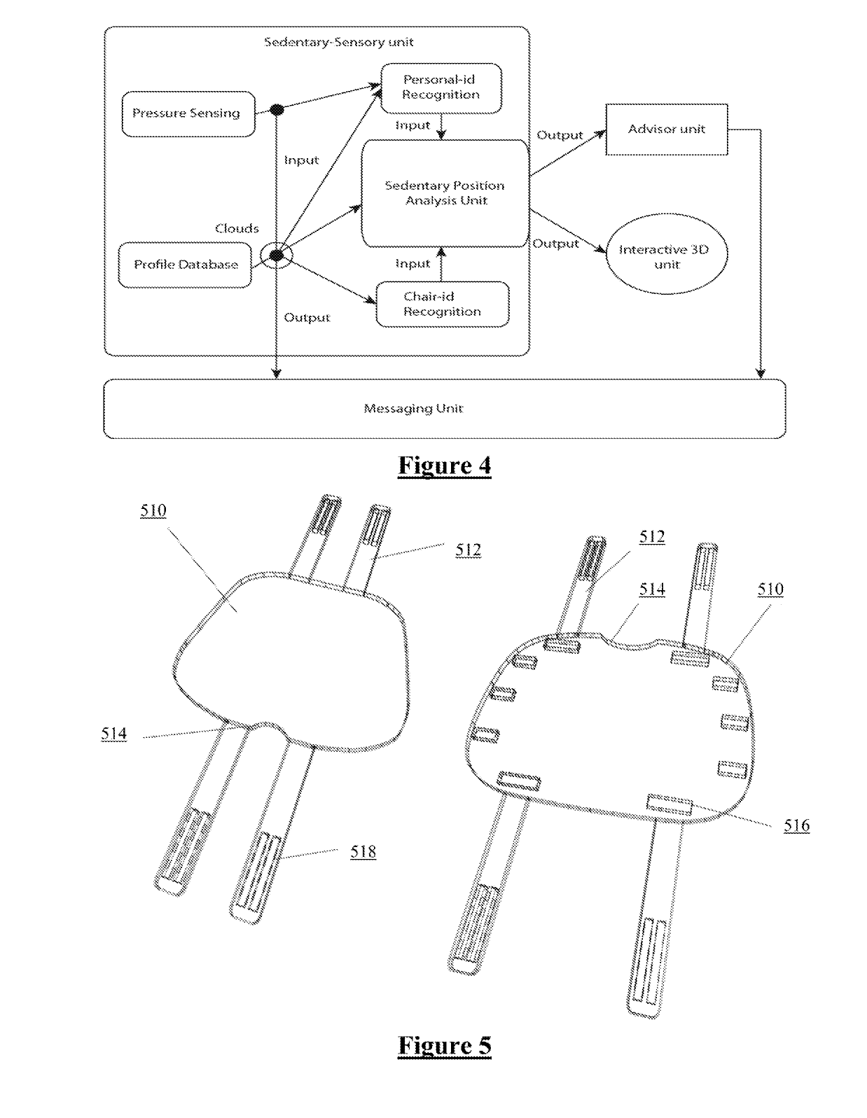

FIG. 4 illustrates another embodiment of the invention further comprising a Personal-id recognition unit. To avoid the confusion of different users sit on the same ‘Sedentary-Sensory Unit’ (e.g. in the form of a chair), the Personal-id Recognition is responsible to recognize ‘who is currently sitting on the chair’. In this embodiment this recognition functionality relies on the user having registered their user profile beforehand.

This Personal-id recognition unit is able to identify different users through distinguishing the differences of pressure input patterns when users are sitting in the same position. By noting the different weight, height and morphology of users, the sensor data returned is processed in order to distinguish one registered user from another. In one scenario, when the user first sits in the chair, the system delivers a message to the user requiring them to sit in a standard straight position until the system recognizes the identity of the user. The system will give feedback as to whether the user's identity could be successfully recognized. The embodiment of FIG. 4 further adapts to changes in user morphology over time by monitoring and recording changes from the sensor data from the chairs in various locations and the user profile can be repeatedly updated over time.

An advantage of the above embodiments is that the sensor and communications components are installed on chairs rather than attached to the user's body

The benefits of some embodiments of this invention might also include the monitoring of unrelieved pressure, monitoring of shear forces and friction, minimizing costs to the health system caused by pressure ulcers, supporting nursing staff to make informed decisions about patient care, ensuring compliance of clinical standards and enhancing the overall quality of care.

In order to detect and distinguish various body positions (in both of the embodiments described here), the following 4 step process is followed:
1. In the software program, create an Array (Array Training Input) for each sensor in order to record the input training value;
2. In calibration, take a ‘snap shot’ of the current user's body position and push the value of each sensor into the corresponding array;
3. To verify the calibration, it's better to repeat the snap shot for n times for the same position, a ‘neutral’ value for the corresponding array will be generated;
4. Click the stop button to finish the training process and go to the monitoring process or calibration step for another body position.

In order to detect and distinguish various position of users' body (i.e. lying on bed), it is necessary to extract the calibrated value of ‘Hotspots’ from the force sensing matrix data, to then in turn estimate the projection of the value on to the graphical expression (i.e. enlarged circles) of user interface.

In order to analyse the value for each sensor, need to follow the following steps:
(variance(Array Pattern Input)_Threshold Pattern)
Pattern Sensor Value=mean(Array Pattern Input)
else
Pattern Sensor Value=DefaultValue
end
   Here according to the real experimental results, the optimal Threshold Pattern=900 and DefaultValue here is set as 15.

In order to select the Hotspots from the force sensing matrix data, the following process need to be followed
1. Create an Array (Array Input) to store the current input value of each sensor locally (i.e. a local .txt file) or upload into the cloud;
2. Compare the input sensor value to the value record in the sensor as follows:
For each sensor where Vep is the value in existing patterns and ThreKP is the threshold of each key points. If the following equation is true,

Vep≧ThreKP
Then pick the sensor as the Hotspot sensor.

In order to use the validation result of the given body position for comparison and reviewing in the later stage, it's necessary to store the validated data into an independent file. Create an Array (Array Pattern) for the pattern (store the value of each sensor but not only the Hotspot sensors) and record the pattern to a local file (i.e. a local .txt file) or upload into the cloud

The projection of array data between the ‘Training Input’ and ‘Snap Shot’ can be estimated by Hotspot Difference for Different Body Parts (HD-DBP) measuring the differences for the same position sensing data under the same body parts (i.e. shoulder, back, waist) as illustrated in Figure xx. This equation serves the turn to all the hotspot sensors

$$\frac{{Σ ( Iv - Pv )}^{2}}{{Σ ( MaxVsen - MinVsen )}^{2}} \leq ValT$$

where Iv is the current input value of the sensor and Pv is the pattern value which is determined through the n times ‘snap short’ process. MaxVsen is the maximum value sensor and MinVsen is the minimum value of the sensor

Here according to the real experimental results, the Threshold-Hotspot and Threshold-Variance are set as 40 and 0.05 separately. The maximum value sensor and minimum value sensor denote the maximum value and the minimum value a sensor can achieve which are set as 150 and 15 respectively.

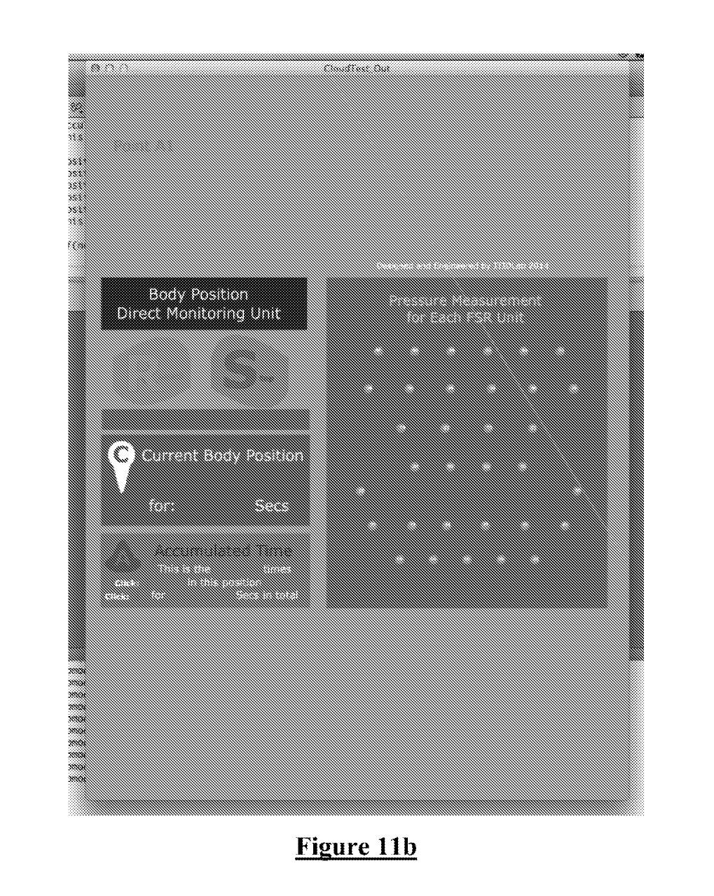

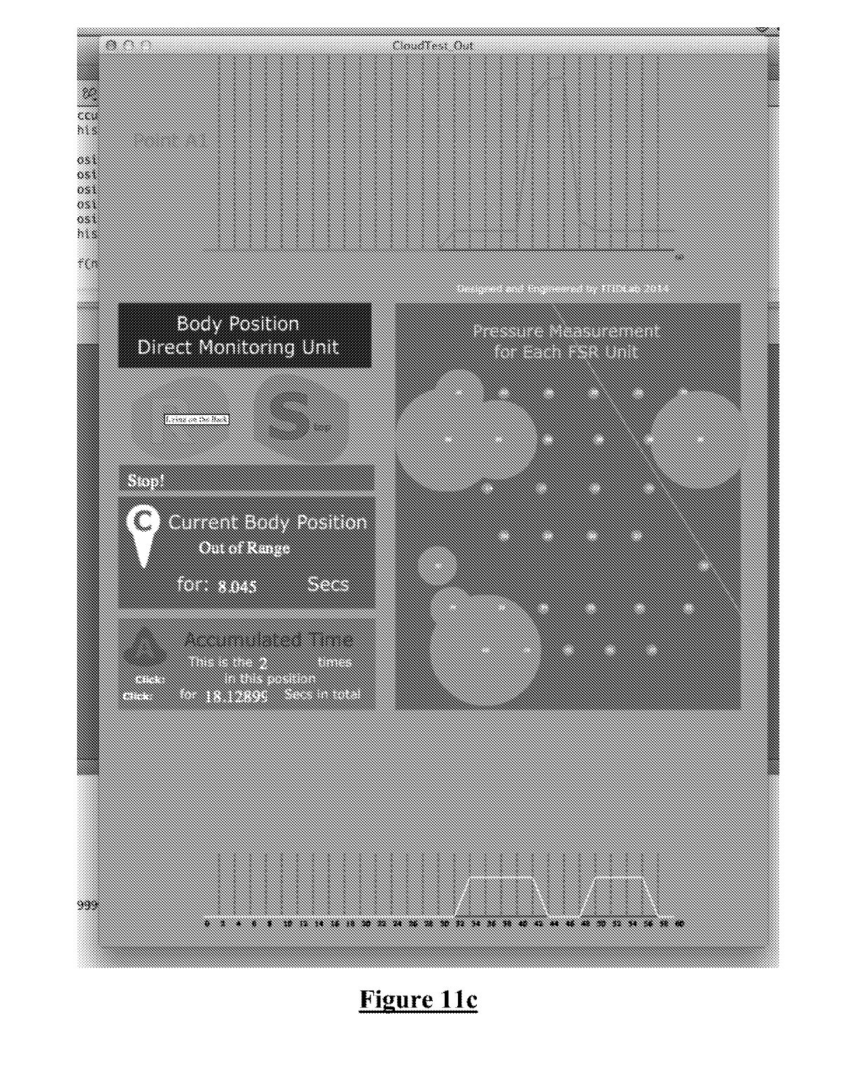

FIG. 11a to 11c show the details of interface design for Direct-Body-Position-Monitoring function of the pressure-sensing embodiment (FIG. 7). Where FIG. 11a illustrates the interface is undertaking Monitoring task after the completion of the Calibration steps; to clarify the Calibration process, FIG. 11b illustrates the initial stage of such process; FIG. 11c illustrates the step right after the Calibration process.

Under a typical Calibration circumstance showing in FIG. 11a, where ‘Record’ button 1110 is used to take a ‘snap shot’ of the pressure inputs of all the sensing units, the amounts of pressure are visualized in real-time 1140. To achieve accurate Calibration results, multiple (normally more than 3) repeated recording in the same body position are required. The time of recording are displayed in the ‘Record Message’ area 1112. After the ‘Recording’ process, the ‘Stop’ button 1111 needs to be clicked, then the system will switch from the Calibration mode to the Monitor mode, a “stop” message will also be displayed in the ‘Record Message’ area 1112

Under the Monitor mode, the current body position is shown in the ‘Current Body Position’ area 1120, for instance, when user is currently lying on his/her back, the ‘lying on the back’ message 1121 will be shown, the ‘lasting time’ will be counted in a millisecond resolution and shown in 1122

Under the Monitor mode, 1130 shows the accumulated information for the current body position. Where 1132 shows how many times the user takes such body position and which last for how long is shown in 1131 with resolution in milliseconds

Under the Monitor mode, the amount of pressure of each sensing unit could be monitored in 1150. Where horizontal scale X represents time span and vertical scale Y represents the value of pressure, higher Y position in the graph shows higher value of the pressure on this sensor

Under the Monitor mode, the activities of any determined and calibrated body position could be monitored in 1160. Where horizontal scale X represents time span and vertical scale Y represents ‘in’ or ‘out’ of this body position, normally high Y position in the graph means ‘on’ and low Y position in the graph means ‘out’

Further embodiments may provide a user interface for Remote-Body-Position-Monitoring function of the pressure-sensing, as discussed in relation to the embodiment of FIG. 7. The targeted user group of such an interface could for example be doctors and clinical practitioners. In the user interface patients' profiles could be selected and current body positions could be monitored. The history of particular body positions could also be monitored through various cloud services, through xively.com as an example.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of monitoring posture of a user, the method comprising:
obtaining first pressure sensor information from a plurality of sensors upon a first item of furniture occupied by the user at a first time;
obtaining first sensor position information defining the relative positions of the sensors upon the first item of furniture;
processing the first pressure sensor information and the first sensor position information to determine a first user posture upon the first item of furniture;
obtaining second pressure sensor information from a plurality of sensors upon a second item of furniture occupied by the user at a second time;
obtaining second sensor position information defining the relative positions of the sensors upon the second item of furniture;
processing the second pressure sensor information and the second sensor position information to determine a second user posture upon the second item of furniture; and
determining a cumulative load upon the user's spine from the first user posture and the second user posture.

2. The method of claim 1 further comprising generating a graphical display indicative of the user's current posture.

3. The method of claim 2 wherein the graphical display comprises an image of a chair of the type currently occupied by the user, and presents an indication of where on the chair the user is placing pressure in their current posture.

4. The method of claim 3 wherein a colour scale is used to indicate variations in pressure applied by the user to the chair.

5. The method of claim 4 wherein the graphical display comprises an image representing a human spine or vertebral column, viewed at an appropriate scale, the image generated in a manner to indicate a current posture of the user's spine as determined from the sensor data.

6. The method of claim 5 wherein a shading colour scale is used to indicate in the image those portions or sections of the spine which are suffering elevated load as a result of the determined posture of the user.

7. The method of claim 6 wherein the sensor position information defining the relative positions of the sensors upon the first item of furniture is stored in a suitable data storage attached to or borne by the item of furniture, with such information being appended to the sensor information.

8. The method of claim 6 wherein the sensor position information defining the relative positions of the sensors upon the first item of furniture comprises a furniture item identifier, which may be used to look up the physical arrangement of sensors on that item of furniture by reference to a furniture ID database of such data, the furniture ID database being separate to the item of furniture.

9. The method of claim 8 wherein each determination of user posture includes an accommodation of individual characteristics of the individual user.

10. The method of claim 9 wherein the user's individual characteristics are held as user profile data in a user profile component of the system.

11. The method of claim 10 wherein the user profile component is stored in the data store of the server computer.

12. The method of claim 11 further comprising delivering output data including or derived from the determined posture and/or cumulative spinal load for review by the user or others.

13. The method of claim 12 wherein the furniture ID database further contains data identifying a display associated with the item of furniture occupied by the user, whereby the messaging system directs information to the associated display.

14. The method of claim 13 wherein the furniture ID database contains data identifying a personal device associated with the user, whereby messaging is directed to the personal device of the user.

15. The method of claim 14 wherein a monitoring period of user posture encompasses both work times, travel times, and rest times, by providing a user's office chair, car seat and armchair with suitable sensors.

16. The method of claim 15 wherein the item of furniture is adjustable, and is equipped with sensors which indicate a current position of each adjustable element of the item of furniture.

17. A system for monitoring posture of a user, the system comprising:
a first item of furniture bearing a first plurality of sensors configured to output first pressure sensor information;
a second item of furniture bearing a second plurality of sensors configured to output second pressure sensor information; and
at least one server computer having a microprocessor and an associated data store which contains instructions executable by the microprocessor such that the server computer is operable to:
process the first pressure sensor information and the first sensor position information to determine a first user posture upon the first item of furniture;
process the second pressure sensor information and the second sensor position information to determine a second user posture upon the second item of furniture; and
determine a cumulative load upon the user's spine from the first user posture and the second user posture.

18. A furniture component for monitoring posture of a user, the furniture component comprising:
a plurality of pressure sensors to detect a pressure exerted by a user occupying the item of furniture,
communications means for communicating the sensor information, and information reflecting the relative positions of the sensors, to at least one server computer configured to determine a user posture upon the item of furniture.

19. A method of monitoring posture of a user, the method comprising:
obtaining pressure sensor information from a plurality of sensors upon an item of furniture occupied by the user;
obtaining sensor position information defining the relative positions of the sensors upon the item of furniture;
processing the pressure sensor information and the sensor position information to determine a user posture upon the item of furniture; and
generating a graphical representation of the user's current posture and displaying the graphical representation to the user on a display associated with the user.

20. A method of identifying a user of an item of furniture, the method comprising:
obtaining pressure sensor information from a plurality of sensors upon the item of furniture occupied by the user;
obtaining sensor position information defining the relative positions of the sensors upon the item of furniture;
processing the pressure sensor information and the sensor position information to determine a morphology of a user currently occupying the item of furniture; and
comparing the determined morphology to records of the morphology of a plurality of individuals in order to determine which one of the plurality of individuals is occupying the item of furniture.
